(19) United States
(12) Patent Application Publication
(10) Pub. No.: US 2006/0276652 A1
(43) Pub. Date: Dec. 7, 2006

(54) Preparation of tadalafil intermediates

(76) Inventors: Ben-Zion Dolitzky, Petach Tiqva (IL); Dov Diller, Jerusalem (IL)

Correspondence Address: KENYON & KENYON LLP, ONE BROADWAY, NEW YORK, NY, 10004, US

(21) Appl. No.: 11/403,582

(22) Filed: Apr. 12, 2006

Related U.S. Application Data
(60) Provisional application No. 60/671,239, filed on Apr. 12, 2005.

(51) Int. Cl. C07D 471/02

(52) U.S. Cl. 546/85

(57) ABSTRACT

Provided is a process for preparing tadalafil intermediates in various solvents. Also provided is a method for converting said intermediates to tadalafil.

### CROSS-REFERENCE TO RELATED APPLICATIONS

This application claims the benefit of Provisional Application Number 60/671,239, filed Apr. 12, 2005, which is incorporated herein by reference.

### FIELD OF THE INVENTION

The invention encompasses processes of preparing tadalafil intermediates in various solvents.

### BACKGROUND OF THE INVENTION

Tadalafil, (6R-trans)-6-(1,3-benzodioxol-5-yl)-2,3,6,7,12,12a-hexahydro-2-methyl-pyrazino[1′,2′:1,6]pyrido[3,4-b]indole-1,4-dione, with the structural formula shown below, is a white crystalline powder. (CAS# 171596-29-5). Tadalafil is a potent and selective inhibitor of the cyclic guanosine monophosphate (cGMP)—specific phosphodiesterase enzyme, PDE5. The inhibition of PDE5 increases the amount of cGMP, resulting in smooth muscle relaxation and increased blood flow. Tadalafil is therefore currently used in the treatment of male erectile dysfunction.

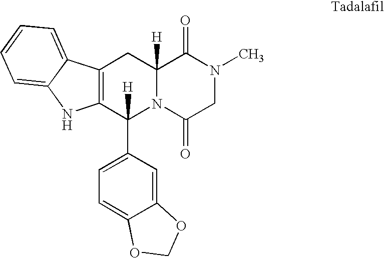

Tadalafil may be prepared via a series of intermediates. One synthesis scheme is illustrated in Scheme 1:

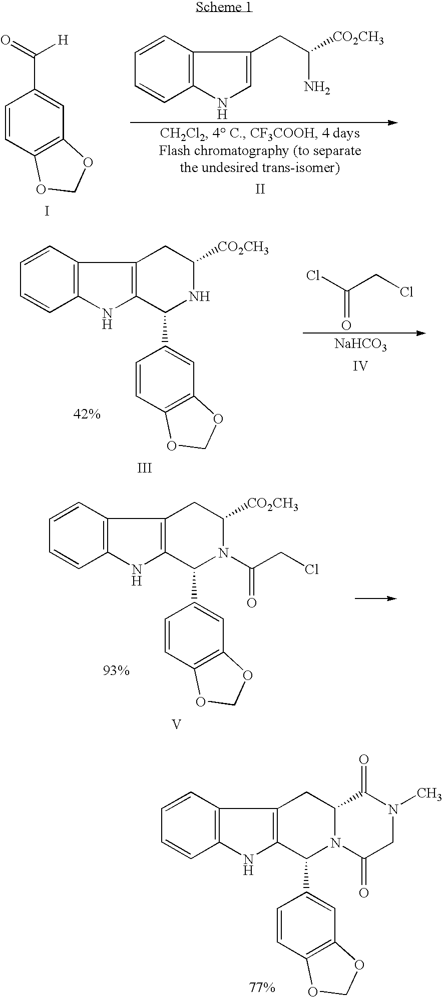

U.S. Pat. No. 5,859,006 describes the synthesis of the tadalafil intermediate (Compound III) from D-tryptophan methyl ester (Compound II) and piperonal (Compound I) using trifluoroacetic acid and dichloromethane, a halogenated solvent. Compound III is then reacted with chloroacetyl chloride (Compound IV) and chloroform, providing another intermediate of tadalafil (Compound V). WO 04/011463 describes a process of preparing tadalafil intermediates from D-tryptophan methyl ester HCl salt and piperonal by refluxing the reagents in isopropyl alcohol; the obtained intermediate is reacted with chloroacetyl chloride and THF, resulting in another intermediate of tadalafil.

Cost effective methods of synthesizing tadalafil utilizing safe reagents are highly desirable.

### SUMMARY OF THE INVENTION

In one aspect, the present invention relates to a process for preparing an intermediate, useful in the preparation of tadalafil, herein referred to as Compound III, having the structural formula shown below, 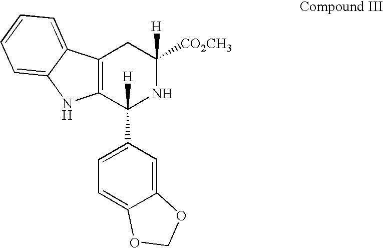
including the steps of: combining D-tryptophan methyl ester or a salt thereof and piperonal with at least one organic reaction solvent selected from the group consisting of alkyl esters of lower carboxylic acids and aromatic hydrocarbons to form a first reaction mixture; combining trifluoroacetic acid with the first reaction mixture to form a second reaction mixture, and maintaining the second reaction mixture at a temperature of about 5° C. to about 90° C. to obtain Compound III.

In another aspect, the present invention comprises preparing Compound III as described above, and converting Compound III to tadalafil.

In yet another aspect, the present invention relates to a process for preparing an intermediate useful in the preparation of tadalafil, and herein referred to as Compound V, having the structural formula shown below, 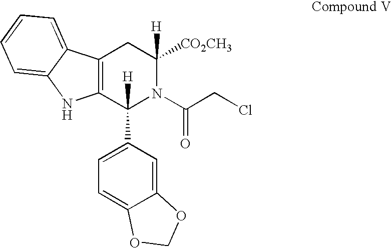
including the steps of: combining Compound III, an organic reaction solvent selected from the group consisting of aromatic hydrocarbon, non cyclic ethers and alkyl esters of lower carboxylic acids and a base to form a first reaction mixture; combining the first reaction mixture with chloroacetyl chloride to form a second reaction mixture; and maintaining the second reaction mixture at a temperature of less than about 1 0C to obtain Compound V.

In yet a further aspect, the present invention comprises preparing Compound V as described above, and converting Compound V to tadalafil.

### DETAILED DESCRIPTION OF THE INVENTION

The invention provides a process of preparing tadalafil intermediate Compound III, having the chemical name cis-methyl 1,2,3,4-tetrahydro-1-(3,4-methylenedioxyphenyl)-9H-pyrido]3,4-b]indole-3-carboxylate, and tadalafil intermediate Compound V (also known as tadalafil chloride—“TDCl”) having the chemical name cis-methyl 1,2,3,4-tetrahydro-2-chloroacetyl-1-(3,4-methylenedioxyphenyl)-9H-pyrido]3,4-b]indole-3-carboxylate. The process of the invention does not use halogenated hydrocarbons.

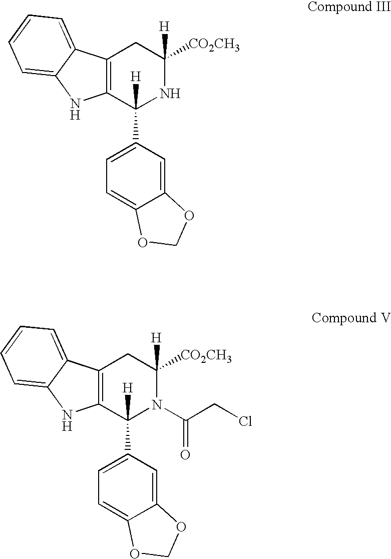

The process of preparing intermediate Compound III includes the steps of combining D-tryptophan methyl ester or a salt thereof and piperonal with at least one organic reaction solvent selected from the group consisting of alkyl esters of lower carboxylic acids, and aromatic hydrocarbons to form a first reaction mixture; combining trifluoroacetic acid with the first reaction mixture to form a second reaction mixture; and maintaining the second reaction mixture at a temperature of about 5° C. to about 90° C. to obtain Compound III.

A preferred salt of D-tryptophan methyl ester is the hydrochloride salt.

The term “alkyl esters of lower carboxylic acids,” as used herein, refers to organic compounds having the general structure R′—COOR″, wherein R′ is a linear or branched alkyl group having from 1 to 6 carbon atoms, and R″ is a linear or branched alkyl group having from 1 to 6 carbon atoms. Preferably, the alkyl group R′ has 1 to 3 carbon atoms. Preferably, the alkyl group R″ has 1 to 4 carbon atoms, more preferably from 1 to 3 carbon atoms. Alkyl esters of lower carboxylic acids preferred for use in the invention include ethyl acetate, propyl acetate, butyl acetate, isopropyl acetate, and isobutyl acetate.

Aromatic hydrocarbons are well known in the art. The aromatic hydrocarbons used in the above process can be any one of benzene, toluene and xylene.

As used herein, the term “room temperature” refers to a temperature range between about 15° C. and 30° C.

Piperonal is used in an amount sufficient to react with D-tryptophan methyl ester, for example, in a stoichiometric amount, or in excess of the amount of D-tryptophan methyl ester. Preferably, piperonal is used in an amount of about 1.0 to about 10.0 molar equivalents to D-tryptophan methyl ester. More preferably, piperonal is used in an amount of about 1.0 to about 1.5 molar equivalents to D-tryptophan methyl ester.

Preferably, the organic reaction solvent used in the process of preparing intermediate Compound III is ethyl acetate. The organic reaction solvent is used in an amount of about 6 to about 100 volumes (volume of reaction solvent-to-weight).

The process of the reaction preferably includes the step of cooling the first reaction mixture, such as in an ice bath, before combining the first reaction mixture with trifluoroacetic acid. Preferably, the first reaction mixture is cooled to a temperature of less than about 10° C., more preferably, to a temperature of less than 3° C. Trifluoroacetic acid is preferably combined in small aliquots, especially dropwise, with the first reaction mixture to form a second reaction mixture. Preferably, trifluoroacetic acid is used in an amount of about 1.0 to about 100.0 molar equivalents.

The second reaction mixture is agitated, for example by stirring, for a reaction time which depends upon, among other things, the scale of the reaction, the size of the equipment used in the reaction, and the type of agitation provided. Reaction time can be determined by one skilled in the art by routine experimentation; for example, by measuring the absence of the limiting reagent using such techniques as HPLC. A reaction time of about 2 hours to about 7 days is typically sufficient. Preferably, the reaction time is about 4 days to about 7 days.

The second reaction mixture is preferably maintained at a temperature of about room temperature or about 30° C. to about 60° C.

The process of the invention optionally includes filtering the second reaction mixture after the reaction time.

Another embodiment of the invention provides a process for preparing tadalafil including preparing Compound III by the process described above, and converting it to tadalafil. The conversion of Compound III to tadalafil may be performed by any method known in the art, such as the one described in U.S. Pat. No. 5,859,006.

In a further embodiment, the invention provides a process for the preparation of tadalafil intermediate Compound V including the steps of: combining Compound III or salt thereof, an organic reaction solvent selected from the group consisting of aromatic hydrocarbon, non-cyclic ethers and alkyl esters of lower carboxylic acids and a base to form a first reaction mixture; combining the first reaction mixture with chloroacetyl chloride to form a second reaction mixture; and maintaining the second reaction mixture at a temperature of less than about 10° C. to obtain Compound V.

Preferably, a salt of Compound III is used to form the first reaction mixture, more preferably the HCl salt of Compound III is used.

Alkyl esters of lower carboxylic acids used are as defined above. Examples of non-cyclic aliphatic ethers include diethyl ether, dipropyl ether, and isopropyl ether.

Preferably, a weak base is used. The term “weak base,” when used herein, refers to an organic base having a pKb of about 2 to about 8, and preferably having a pKb of about 3 to about 7. Weak bases include, but are not limited to, C₁₋₆ mono-di- or tri-alkyl amines, wherein the alkyl groups may be same or different, and carbonate salts of Group I or Group II metals, in particular Na, K, Li, etc. Preferably, the weak base used in preparing intermediate Compound V is triethylamine or potassium carbonate. The weak base is present in an amount of about 1.0 to about 10.0 molar equivalents to Compound III. Preferably, the weak base is present in an amount of about 3.0 to about 10.0 molar equivalents to Compound III.

Organic reaction solvents useful for the preparation of Compound V in this embodiment of the invention include aromatic hydrocarbons, alkyl esters of lower carboxylic acids and methyltert-butylether, or combinations of two or more of these. The organic reaction solvent in this embodiment of the invention is preferably ethyl acetate or toluene. Preferably, the organic reaction solvent is used in an amount of about 1 to about 10 by volume of Compound III. More preferably, the organic reaction solvent is used in an amount of about 3 to about 10 by volume of Compound III.

The first reaction mixture is optionally cooled in an ice bath before combining with the chloroacetyl chloride to form a second reaction mixture. In a preferred embodiment of the invention, the first reaction mixture is cooled to about 5° C. before combining with chloroacetyl chloride. Chloroacetyl chloride can be and preferably is dissolved in the organic reaction solvent used to form the first reaction mixture, and the resulting combination is preferably combined dropwise with the first reaction mixture. Chloroacetyl chloride is preferably used in an amount of about 1 to about 8 molar equivalents to Compound III. More preferably, chloroacetyl chloride is present in an amount of about 1 to about 5 molar equivalents to Compound III.

The second reaction mixture is preferably maintained at about 5° C. for a reaction time. The reaction time depends on, among other things, the scale of the reaction, the size of the equipment used in the reaction, and the type of agitation provided. Reaction time can be determined by one skilled in the art by routine experimentation; for example, by measuring the absence of the limiting reagent using such techniques as HPLC. A reaction time of about 5 minutes to about 4 hours is typically sufficient. Preferably, the reaction time is about 15 minutes to about two hours.

The process of the invention optionally includes stirring the second reaction mixture at about room temperature after the reaction time. Preferably, the second reaction mixture is stirred at about room temperature from about 20 minutes to about 10 hours, more preferably, for about two hours. The second reaction mixture may optionally be concentrated, stirred in isopropyl alcohol and water, filtered, and dried.

Another embodiment of the invention provides a process for preparing tadalafil including preparing Compound V by the process described above, and converting it to tadalafil. The conversion of Compound V to tadalafil may be performed by any method known in the art, such as the one described in U.S. Pat. No. 5,859,006.

The present invention is, in certain of its embodiments, exemplified by the following non-limiting examples.

### EXAMPLES

### Example 1

### Synthesis of Intermediate Compound III in Ethyl Acetate at Room Temperature

D-tryptophan methyl ester (10.9 g, 50 mmol), ethyl acetate (200 ml), and piperonal (7.9 g, 52.06 mmol) were combined to form a reaction mixture at room temperature. The reaction mixture was stirred and cooled in an ice bath. Trifluoroacetic acid (7.7 ml, 100 mmol) was added dropwise to the reaction mixture. The reaction mixture was removed from the ice bath and stirred at room temperature for about 7 days. The reaction mixture was then filtered. Compound III was obtained in a yield of 75%.

### Example 2

### Synthesis of Intermediate Compound III in Ethyl Acetate at About 45° C. to About 50° C.

D-tryptophan methyl ester (5.0 g, 23 mmol), ethyl acetate (200 ml), and piperonal (3.9 g, 26 mmol) were combined to form a reaction mixture at room temperature. The D-tryptophan methyl ester did not dissolve. The reaction mixture was stirred and cooled in an ice bath. Trifluoroacetic acid (3.8 ml) was added dropwise to the reaction mixture. The reaction mixture was removed from the ice bath and stirred at about 45° C. to about 50° C. for about 7 days. The reaction mixture was then filtered. Compound III was obtained in a yield of 32%.

### Example 3

### Synthesis of Intermediate Compound V in THF and Triethylamine

Intermediate Compound III .HCl (3 g, 7.75 mmol), THF (12 ml), and triethylamine (2 g, 18.55 mmol) were combined to form a reaction mixture. The reaction mixture was stirred and cooled in an ice/salt bath to a temperature of about 5° C. Chloroacetyl chloride (1.22 g, 10.8 mmol) dissolved in THF (2 ml) was added dropwise to the reaction mixture over a period of about 15 minutes while the temperature was maintained at less than about 10° C. After an additional 15 minutes, the reaction mixture was taken out of the ice bath and stirred at room temperature for about 30 minutes. The reaction mixture was then concentrated under vacuum. Isopropyl alcohol (12 ml) and water (6 ml) were added to the reaction mixture and the reaction mixture was stirred for about 2 hours at room temperature. The reaction mixture was filtered and dried for about 2 hours, yielding Compound V (2.15 g, 65% yield).

### Example 4

### Synthesis of Intermediate Compound V in Toluene and Triethylamine

Intermediate Compound III HCl (3 g, 7.75 mmol), toluene (12 ml), and triethylamine (2 g, 18.55 mmol) were combined to form a reaction mixture. The reaction mixture was stirred and cooled in an ice/ salt bath to a temperature of about 5° C. Chloroacetyl chloride (1.22 g, 10.8 mmol) dissolved in toluene (2 ml) was added dropwise to the reaction mixture over a period of about 15 minutes while the temperature was maintained at less than about 10° C. After an additional 15 minutes, the reaction mixture was taken out of the ice bath and stirred at room temperature for about 30 minutes. The reaction mixture was then concentrated under vacuum. Isopropyl alcohol (12 ml) and water (6 ml) were added to the reaction mixture and the reaction mixture was stirred for about 2 hours at room temperature. The reaction mixture was filtered and dried for about 2 hours, yielding Compound V (2.22 g, 67% yield).

### Example 5

### Synthesis of Intermediate Compound V in MTBE and Triethylamine

Intermediate Compound III HCl (3 g, 7.75 mmol), MTBE (12 ml), and triethylamine (2 g, 18.55 mmol) were combined to form a reaction mixture. The reaction mixture was stirred and cooled in an ice/ salt bath to a temperature of about 5° C. Chloroacetyl chloride (1.22 g, 10.8 mmol) dissolved in MTBE (2 ml) was added dropwise to the reaction mixture over a period of about 15 minutes while the temperature was maintained at less than about 10° C. After an additional 15 minutes, the reaction mixture was taken out of the ice bath and stirred at room temperature for about 65 minutes. The reaction mixture was then concentrated under vacuum. Isopropyl alcohol (12 ml) and water (6 ml) were added to the reaction mixture and the reaction mixture was stirred for about 2 hours at room temperature. The reaction mixture was filtered and dried for about 2 hours, yielding Compound V (2.01 g, 61% yield).

### Example 6

### Synthesis of Intermediate Compound V in Ethyl Acetate and Triethylamine

Intermediate Compound III HCl (3 g, 7.75 mmol), ethyl acetate (12 ml), and triethylamine (2 g, 18.55 mmol) were combined to form a reaction mixture. The reaction mixture was stirred and cooled in an ice/salt bath to a temperature of about 5° C. Chloroacetyl chloride (1.22 g, 10.8 mmol) dissolved in ethyl acetate (2 ml) was added dropwise to the reaction mixture over a period of about 15 minutes while the temperature was maintained at less than about 10° C. After an additional 15 minutes, the reaction mixture was taken out of the ice bath and stirred at room temperature for about 70 minutes. The reaction mixture was then concentrated under vacuum. Isopropyl alcohol (12 ml) and water (6 ml) were added to the reaction mixture and the reaction mixture was stirred for about 2 hours at room temperature. The reaction mixture was filtered and dried for about 2 hours, yielding Compound V (3.21 g, 97% yield).

### Example 7

### Synthesis of Intermediate Compound V in Toluene and Potassium Carbonate

Intermediate Compound III HCl (3 g, 7.75 mmol), toluene (12 ml), and potassium carbonate (2 g, 18.55 mmol) were combined to form a reaction mixture. The reaction mixture was stirred and cooled in an ice/ salt bath to a temperature of about 5° C. Chloroacetyl chloride (1.22 g, 10.8 mmol) dissolved in toluene (2 ml) was added dropwise to the reaction mixture over a period of about 15 minutes while the temperature was maintained at less than about 10° C. After an additional 15 minutes, the reaction mixture was taken out of the ice bath and stirred at room temperature for about 35 minutes. The reaction mixture was then concentrated under vacuum. Isopropyl alcohol (12 ml) and water (6 ml) were added to the reaction mixture and the reaction mixture was stirred for about 2 hours at room temperature. The reaction mixture was filtered and dried for about 2 hours, yielding Compound V (0.22 g, 3.7% yield).

### Example 8

### Synthesis of Intermediate Compound V in MTBE and Potassium Carbonate

Intermediate Compound III .HCl (3 g, 7.75 mmol), MTBE (12 ml), and potassium carbonate (2 g, 18.55 mmol) were combined to form a reaction mixture. The reaction mixture was stirred and cooled in an ice/salt bath to a temperature of about 5° C. Chloroacetyl chloride (1.22 g, 10.8 mmol) dissolved in MTBE (2 ml) was added dropwise to the reaction mixture over a period of about 15 minutes while the temperature was maintained at less than about 10° C. After an additional 15 minutes, the reaction mixture was taken out of the ice bath and stirred at room temperature for about 45 minutes. The reaction mixture was then concentrated under vacuum. Isopropyl alcohol (12 ml) and water (6 ml) were added to the reaction mixture and the reaction mixture was stirred for about 2 hours at room temperature. The reaction mixture was filtered and dried for about 2 hours, yielding Compound V (0.42 g).

### Example 9

### Synthesis of Intermediate Compound V in Ethyl Acetate and Potassium Carbonate

Intermediate Compound III .HCl (3 g, 7.75 mmol), ethyl acetate (12 ml), and potassium carbonate (2 g, 18.55 mmol) were combined to form a reaction mixture. The reaction mixture was stirred and cooled in an ice/salt bath to a temperature of about 5° C. Chloroacetyl chloride (1.22 g, 10.8 mmol) dissolved in ethyl acetate (2 ml) was added dropwise to the reaction mixture over a period of about 15 minutes while the temperature was maintained at less than about 10° C. After an additional 15 minutes, the reaction mixture was taken out of the ice bath and stirred at room temperature for about 2 hours. The reaction mixture was then concentrated under vacuum. Isopropyl alcohol (12 ml) and water (6 ml) were added to the reaction mixture and the reaction mixture was stirred for about 2 hours at room temperature. The reaction mixture was filtered and dried for about 2 hours, yielding Compound V (0.72 g).

What is claimed is:

1. A process of preparing Compound III having the formula 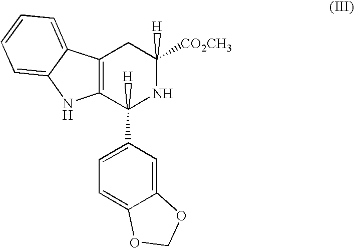
comprising:
a) combining D-tryptophan methyl ester or a salt thereof and piperonal with at least one organic reaction solvent selected from the group consisting of alkyl esters of lower carboxylic acids and aromatic hydrocarbons to form a first reaction mixture;
b) combining trifluoroacetic acid with the first reaction mixture to form a second reaction mixture; and
c) maintaining the second reaction mixture at a temperature of about 5° C. to about 90° C. to obtain Compound III.

2. The process of claim 1, wherein the hydrochloride salt of D-tryptophan methyl ester is used in step a).

3. The process of claim 1, wherein the organic reaction solvent is selected from the group consisting of: benzene, toluene, xylene, ethyl acetate, propyl acetate, butyl acetate, isopropyl acetate, and isobutyl acetate.

4. The process of claim 3, wherein the organic reaction solvent is selected from the group consisting of ethyl acetate, propyl acetate, butyl acetate, isopropyl acetate, and isobutyl acetate.

5. The process of claim 4, wherein the organic reaction solvent is ethyl acetate.

6. The process of claim 1, wherein the piperonal is present in an amount of about 1.0 to about 10.0 molar equivalents to D-tryptophan methyl ester.

7. The process of claim 6, wherein the piperonal is present in an amount of about 1.0 to about 1.5 molar equivalents to D-tryptophan methyl ester.

8. The process of claim 1, wherein the organic reaction solvent is used in an amount of about 6 to about 100 volumes (volume-to-weight).

9. The process of claim 1, further comprising the step of cooling the first reaction mixture prior to step b) to a temperature of less than about 10° C.

10. The process of claim 9, wherein the cooling is to a temperature of less than about 3° C.

11. The process of claim 1, wherein the trifluoroacetic in step b) is combined dropwise with the first reaction mixture.

12. The process of claim 1, wherein trifluoroacetic acid is combined in an amount of about 1.0 to about 100.0 molar equivalents.

13. The process of claim 1, wherein the second reaction mixture is maintained with agitation for about 2 hours to about 7 days.

14. The process of claim 13, wherein the second reaction mixture is maintained with agitation for about 4 days to about 7 days.

15. The process of claim 1, wherein the temperature in step c) is about room temperature to about 60° C.

16. In a process for preparing tadalafil via compound III, the steps of:
a) combining D-tryptophan methyl ester or a salt thereof and piperonal with at least one organic reaction solvent selected from the group consisting of alkyl esters of lower carboxylic acids and aromatic hydrocarbons to form a first reaction mixture;
b) combining trifluoroacetic acid with the first reaction mixture to form a second reaction mixture; and
c) maintaining the second reaction mixture at a temperature of about 5° C. to about 90° C. to obtain Compound III.

17. A process for preparing Compound V of the formula 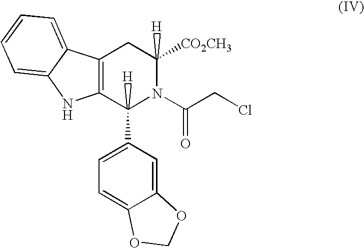
Comprising the steps of:
a) combining Compound III or a salt thereof, an organic reaction solvent selected from the group consisting of aromatic hydrocarbons, non cyclic ethers and alkyl esters of lower carboxylic acids; and a base to form a first reaction mixture;
b) combining the first reaction mixture with chloroacetyl chloride to form a second reaction mixture; and
c) maintaining the second reaction mixture at a temperature of less than about 10° C. to obtain Compound V.

18. The process of claim 17, wherein a salt of Compound III is combined in step a).

19. The process of claim 18, wherein the salt of Compound III is the HCl salt.

20. The process of claim 17, wherein the base is a weak base.

21. The process of claim 20, wherein the weak base is selected from the group consisting of triethylamine and potassium carbonate.

22. The process of claim 17, wherein the base is present in an amount of about 1.0 to about 10.0 molar equivalents to Compound III.

23. The process of claim 22, wherein the base is present in an amount of about 3.0 to about 10.0 molar equivalents to Compound III.

24. The process of claim 17, wherein the organic reaction solvent is selected from the group consisting of methyltert-butylether, ethyl acetate and toluene.

25. The process of claim 24, wherein the organic reaction solvent is selected from the group consisting of ethyl acetate and toluene.

26. The process of claim 17, wherein the organic reaction solvent is used in an amount of about 1 to about 10 volumes of Compound III.

27. The process of claim 26, wherein the organic reaction solvent is used in an amount of about 3 to about 10 volumes of Compound III.

28. The process of claim 17, further comprising the step of cooling the first reaction mixture prior to step b) to a temperature of less than about 5° C.

29. The process of claim 17, wherein the chloroacetyl chloride in step b) is combined in the organic reaction solvent used to form the first reaction mixture in step a).

30. The process of claim 17, wherein the chloroacetyl chloride in step b) is combined dropwise with the first reaction mixture.

31. The process of claim 17, wherein the chloroacetyl chloride is combined in an amount of about 1 to about 8 equivalents to Compound III.

32. The process of claim 31, wherein the chloroacetyl chloride is present in an amount of about 1 to about 5 molar equivalents to Compound III.

33. The process of claim 17, wherein the second reaction mixture is maintained for a reaction time at a temperature of about 5° C.

34. The process of claim 33, wherein the reaction time is about 5 minutes to about 4 hours.

35. The process of claim 34, wherein the reaction time is about 15 minutes to about two hours.

36. The process of claim 17, wherein the second reaction mixture is maintained after a reaction time, while stirring, at about room temperature.

37. The process of claim 17, wherein the second reaction mixture is maintained for about 20 minutes to about 10 hours.

38. In a process for preparing tadalafil via compound V, the steps of:
a) combining Compound III or salt thereof, an organic reaction solvent selected from the group consisting of aromatic hydrocarbons, non cyclic ethers and alkyl esters of lower carboxylic acids, and a base to form a first reaction mixture;
b) combining the first reaction mixture with chloroacetyl chloride to form a second reaction mixture; and
c) maintaining the second reaction mixture at a temperature of less than about 10° C. to obtain Compound V.
